(19) United States
(12) Patent Application Publication
(10) Pub. No.: US 2007/0083206 A1
(43) Pub. Date: Apr. 12, 2007

(54) Self drilling and tapping bone screw

(75) Inventor: Harrison Du, Tucheng City (TW)

Correspondence Address: NIKOLAI & MERSEREAU, P.A., 900 SECOND AVENUE SOUTH, SUITE 820, MINNEAPOLIS, MN, 55402, US

(73) Assignee: MICROWARE PRECISION CO., LTD., Tucheng City (TW)

(21) Appl. No.: 11/231,959

(22) Filed: Sep. 21, 2005

(51) Int. Cl. A61B 17/58

(52) U.S. Cl. 606/73

(57) ABSTRACT

A self drilling and tapping bone screw for orthodontic, oral, facial or cranial surgery in bone tissue is disclosed. The bone screw includes a screw head and a body portion. The body portion is provided with a thread and a body tip. There are multiple cutting edges formed by recessed grooves on the body tip. Extending from behind each cutting edge, a relief plane is formed with a relief angle inclined backward from the surface of cut. In the process of screwing in, the relief plane and bone tissue surface of cut will not have area contact such that they will not adhere to each other. The cutting face in the recessed groove can also be inclined with a rake angle to the plane formed by the bone screw axis and radius. The drilling resistance is thus reduced tremendously, so is the required twisting torque to drilling, tapping and screwing in. Also, the possibility of a breakage of the bone screw in the screwing-in process decreases.

### FIELD OF THE INVENTION

The present invention relates to medical apparatus, and more particularly to a bone screw which provides good self drilling and tapping ability into bone tissue for use in orthodontic, oral, facial or cranial surgery.

### BACKGROUND OF THE INVENTION

Conventionally, the self drilling and tapping bone screw, as shown in FIG. 1, for the surgery to place in with hand power of medical personnel, has no design of cutting edges because of its small size, mostly less than 3 mm in diameter. Since the torque to insert the screw (1) into bone tissue has to be small, the tip portion (11) of the screw body is gradually tapered to a point like a needle tip to reduce the torque required. Although such designed screw can reduce the resistance to screw in, the strength of tip portion is thus reduced to easily cause breakage of the screw. For further improvement, cutting edges are provided by cutting grooves in the tip portion, but the required torque to place in the bone screw is still not low enough to be completely satisfied in a self drilling and tapping process.

Despite the existing art of self drilling and tapping bone screws, there remains yet a need for a bone screw which is self drilling and tapping to be placed in accurately with very low torque. It is to the provision of such a bone screw to have advantages of accurate positioning, easy drilling, smooth tapping and reliable tight-fitting. that this invention is primarily directed.

### SUMMARY OF THE INVENTION

The objective of the present invention is to provide a self drilling and tapping bone screw which can be placed into bone tissue easily and accurately for use in orthodontic, oral, facial or cranial surgery.

The bone screw includes a screw head and body portion. The screw head is used for a hand tool to fit for screwing in and is exposed outside of bone tissue for wire binding or part mounting. The screw body is a shaft provided with a thread and includes a tip portion. The tip portion is provided with several cutting edges which are formed by recessed grooves. Each cutting edge is characterized by extending a relief face right behind the cutting edge such that the relief face forming a backward relief angle with the tangent plane of the surface of cut. Furthermore, the cutting face of the recessed groove is inclined with a rake angle to the plane formed by the bone screw axis and radius.

In the drilling and screwing process for the bone screw, the tip portion touches the drilled surface of bone tissue exclusively on cutting edges instead of fragments of cone areas for a tip portion without a design of a relief face. Thus, a clearance is kept between the screw tip behind each cutting edge and the drilled surface, the adherence of bone tissue to the drilling end will improve. Such that the drilling resistance is lower and that the required torque for drilling and screwing is lower than that without a relief face design. Lower required torque to place in the screw means easier operation, more accurate positioning and lower possibility of a screw breakage. Therefore, this bone screw is able to have the advantages of accurate positioning, easy drilling, smooth tapping and reliable tight-fitting in such surgical operations.

### BRIEF DESCRIPTION OF THE DRAWINGS

FIG. 1 is a side view of a conventional self drilling and tapping bone screw;

FIG. 2 is a side view of the self drilling and tapping bone screw according to the present invention;

FIG. 3 is a side view of the tip portion enlarged from a side view of the self drilling and tapping bone screw according to the present invention.

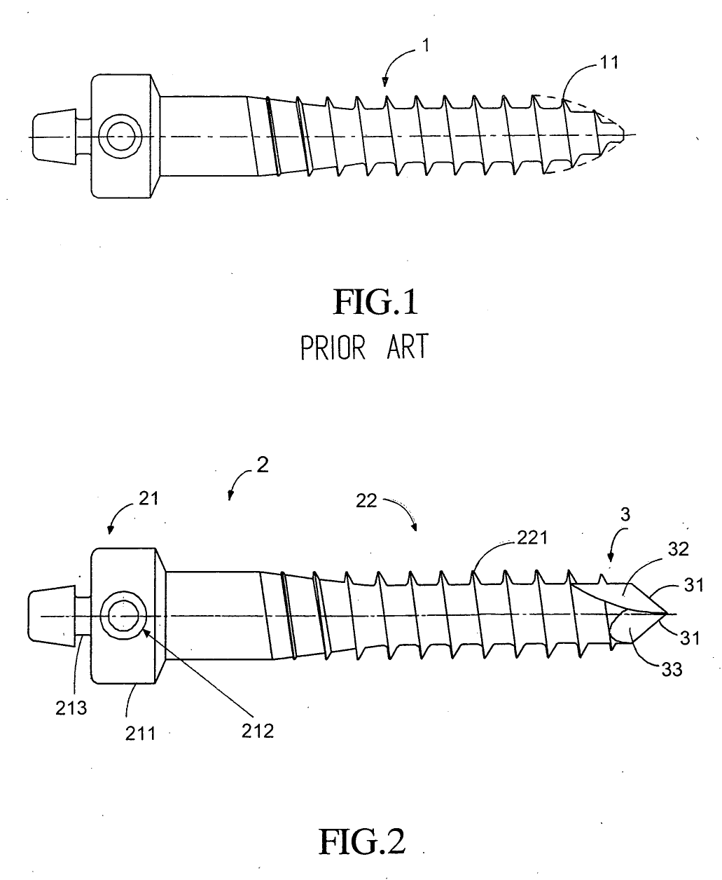

### DETAILED DESCRIPTION OF THE PREFERRED EMBODIMENT

The invention can be further understood by the present detail, which may be read in view of the illustrative drawings of a preferred embodiment.

As illustrative in FIG. 2, the shown self drilling and tapping bone screw (2) is designed to be used in orthodontic operation. The bone screw comprises a screw head (21) and a screw body (22). The screw head (21) is provided with a four-faced stud (211) for tool fitting to apply torque on the screw. After the screw being placed into bone tissue, the screw head (21) will be exposed outside of bone tissue. The four-faced stud (211) is provided with a through hole (212) for wire binding and a circular groove (213) for mounting a spring by protruding above the top face of the four-faced stud (211).

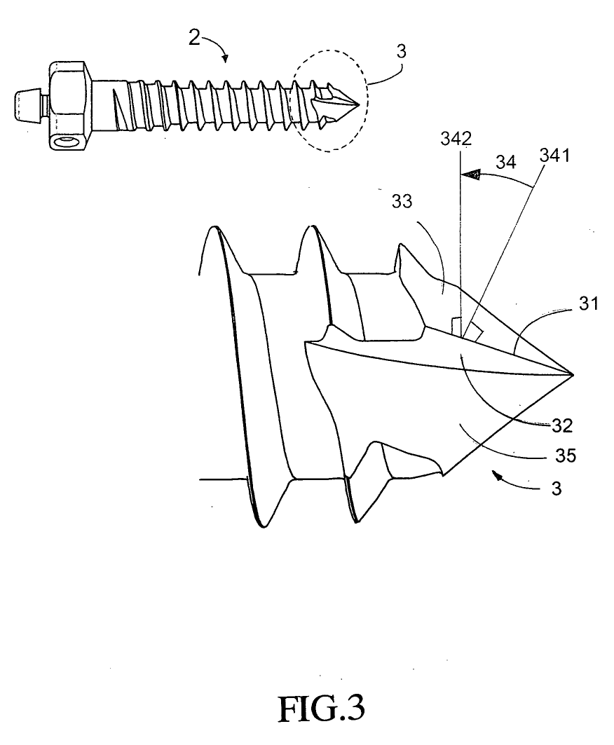

The screw body (22), for screwing into bone tissue, is provided with a shaft covered with a thread (221) and a body tip (3). As shown in FIG. 2, the tip of the screw in includes two cutting edges (31). As shown in FIG. 3, a cutting edge (31) is formed by the intersection line of a cutting face (32) and relief face (33). The cutting face (32), which is on the plane formed by the axis and the radius of the screw, is one of the two walls (32, 35) forming a groove to provide a cutting edge on the tip.

The cutting edge (31) forms a cone shaped surface of cut by rotating around the screw axis. By extending a plane behind a cutting edge (31), a relief face (33) is provided with a relief angle (34), which is formed by retracting an angle from the tangent plane of the cut surface at the cutting edge (31). The relief angle (34) also can be understood as the perpendicular line (341) to the cutting face at a point on the cutting edge rotates inward cone surface of cut to line (342) which is on the relief face perpendicular to the cutting edge. The line (342) and the cutting edge (31) form the relief plane (33). The relief plane extends to intersect a wall, which is not a cutting face, of a groove for another cutting edge. With the existence of the relief angle, the screwing in process performs with a good drilling function. If the cutting face (32) is so designed that face (32) would not be on the plane formed by the screw axis and radius but inclined with a rake angle by pushing the cutting edge (31) forward or pushing the groove bottom of the cutting face backward, and the cutting edge (31) will cut into bone tissue before other part of the cutting face. The drilling conditions will be further improved.

In the process of drilling and tapping of the bone screw, the portion of the screw tip behind each cutting edge keeps a clearance because of the existence of the relief angle between the relief face and the tangent plane of the surface of cut. There is no area contact between bone screw tip and bone tissue except on cutting edges, such that the condition of adherence of the screw to the bone tissue is greatly improved. Consequently, the drilling resistance is lower that the torque for drilling and screwing in is lower than that without relief face design. In the surgery of orthodontics, therefore, this bone screw is able to provide a screwing in process with accurate positioning, easy drilling, smooth tapping and reliable tight-fitting.

What is claimed is:

1. A self drilling and tapping bone screw for screwing into bone tissue, said bone screw comprising:
a screw head; and
a screw body extending from said screw head and including a pointed tip portion, said screw body being provided with thread and said tip portion being provided with a plurality of cutting edges formed by recessed grooves, and said cutting edge characterized by
extending a relief face right behind said cutting edge such that said relief face forming a backward relief angle with the tangent plane of the surface of cut formed by rotating said cutting edge and said tip portion touching the drilled hole formed by said surface of cut exclusively on said cutting edges.

2. A self drilling and tapping bone screw as claimed in claim 1, wherein said recessed groove includes a cutting face, and said cutting face is inclined with a rake angle to the plane formed by the axis and radius of said bone screw.
